(19) United States
(12) Patent Application Publication
(10) Pub. No.: US 2013/0171711 A1
(43) Pub. Date: Jul. 4, 2013

(54) PROCESS FOR OBTAINING DINITROGEN MONOXIDE (N20)

(71) Applicants: Thomas Finke, Ettlingen (DE); Andreas Mattern, Panaji (IN); Dominic Lingenfelser, Hambruecken (DE)

(72) Inventors: Thomas Finke, Ettlingen (DE); Andreas Mattern, Panaji (IN); Dominic Lingenfelser, Hambruecken (DE)

(21) Appl. No.: 13/807,968

(22) PCT Filed: May 11, 2011
(86) PCT No.: PCT/EP11/57571

§ 371 Date: Mar. 20, 2013

(30) Foreign Application Priority Data
Jul. 7, 2010 (DE) 10-2010-031.075.1

(51) Int. Cl. C12P 3/00

(52) U.S. Cl. CPC C12P 3/00
USPC 435/168

(57) ABSTRACT

In a method for obtaining dinitrogen monoxide by microbiological or enzymatic processes from nitrogen-containing substances, the microorganisms, bacteria, archaea, eukaryotes, fungi, parasites, phages, cells, cell fractions or membrane fractions, and/or enzymes, and/or a combination thereof to be used in this context are selected, or manipulated or partly or entirely reversibly and/or irreversibly inhibited by suitable actions, or the corresponding microbiological or enzymatic processes are controlled, for example, by way of suitable process conditions, so that, in part or entirely, dinitrogen monoxide (N₂O) is formed from the nitrogen-containing compounds of the nitrogen-containing substances.

### BACKGROUND OF THE INVENTION

1. Field of the Invention

The present invention relates to a method for obtaining dinitrogen monoxide (N₂O), also called nitrous oxide, by microbiological or enzymatic processes from nitrogen-containing substances, in particular from biomass and/or wastes and/or wastewaters and/or further substances that contain nitrogen-containing compounds, in particular ammonium compounds.

2. Description of the Related Art

Nitrogen-containing substances are, according to the present invention, for example industrial or household wastewaters such as those purified in sewage treatment plants, in particular municipal sewage treatment plants. In the sewage treatment plants, microorganisms are usually used for this purpose. In a staged oxidation process with the participation of oxygen, in particular using so-called oxidizers, e.g. ammonium oxidizers and nitrite oxidizers, firstly ammonium and further nitrogen-containing compounds are oxidized to nitrite ions or nitrate ions. This reaction sequence is referred to in the literature as “nitrification.” The corresponding underlying chemical reactions are, in this context, usually catalyzed enzymatically. Enzymes that play a role in this connection are, for example, the monooxygenases, hydroxylamine oxidoreductases, and nitrite oxidases. A disadvantage of this method is that the nitrification process step, in particular the introduction or “blowing” of oxygen or air into the wastewater, is very energy-intensive. The nitrification process step is usually controlled so that the nitrogen component of all nitrogen-containing substances becomes oxidized as completely as possible to nitrate ions. The process segment, in terms of time or location, in which air or oxygen is blown into the treatment tank is usually referred to as an “aerobic” stage.

A subsequent reduction reduces the nitrate ions that are present, or the nitrite ions that are present in part, in staged fashion to dinitrogen (N₂). This escapes into the atmosphere. In sewage treatment plants, microorganisms are usually used for this reduction. The reduction of nitrite ions or nitrate ions is usually catalyzed, by analogy with the oxidation of the nitrogen-containing substances, by suitable enzymes of the microorganisms. As a rule, nitrate ions are reduced by nitrate reductases to nitrite ions, nitrite ions by nitrite reductases to nitrogen monoxide, the latter by nitrogen monoxide reductases to dinitrogen monoxide, and the latter by dinitrogen monoxide reductases to dinitrogen (N₂). This procedure is generally also referred to as “denitrification.” The denitrification process step is usually controlled so that the nitrate ions and/or nitrite ions that are present are reduced as completely as possible to dinitrogen. The process segment, in terms of time or location, in which no air or oxygen is blown into the treatment tank is usually referred to as an “anaerobic” stage.

Suitable microorganisms are used for the above-described biological oxidation of ammonium ions or further nitrogen-containing compounds of nitrogen-containing substances to nitrite ions or nitrate ions, and for the above-described subsequent biological reduction of nitrate ions and/or nitrite ions. These microorganisms are generally also referred to as “nitrifiers” or “denitrifiers,” respectively. Depending on the process step, both heterotrophic and autotrophic bacteria, lithoautotrophic or chemolithoautotrophic microorganisms, fungi, parasites, or phages are suitable for this. In general, bacteria of the Nitrosococcus genus, as well as Nitrosovibrio, Nitrosomonas, Nitrosospira, and Nitrosolobus, as well as Nitrobacter, are used as nitrifiers. The ability to denitrify is in general widespread within the prokaryotes. Suitable autotrophic bacteria are, for example, Paracoccus denitrificans or Thiobacillus denitrificans. Heterotrophic bacteria used are, for example, Pseudomonas stutzeri. The microorganisms Pseudomonas poutida, Pseudomonas fluorescens, and Alcaligenes faecalis, as well as further representatives of the genera Flavobacteria, Arthrobacter, Achromobacter, Alcaligenes, Moraxella, Pseudomonas, and Hyphomicrobium are also often used. The result aimed at with the use a corresponding combination of nitrifiers and denitrifiers, in the context of wastewater treatment and treatment of further nitrogen-containing substances, is as a rule complete oxidation of the nitrogen component of the nitrogen-containing compounds to nitrate ions and/or nitrate ions, as well as subsequent complete reduction of the nitrate ions and/or nitrite ions to dinitrogen (N₂).

The process sequence of nitrification and denitrification is realized differently in a variety of established methods. For example, alternatively to the sequence described above (i.e. first carrying out nitrification and then implementing denitrification), the anaerobic stage in which denitrification partly and/or entirely takes place can precede the aerobic stage in which nitrification proceeds partly and/or entirely. In both process sequence variants, maximally complete final conversion of the nitrogen component of the nitrogen-containing compounds into dinitrogen (N₂) is achieved by partial recycling of the wastewater, or the water/sludge mixture, that leaves the respectively preceding stage. It is furthermore possible to implement wastewater purification in a tank cascade, i.e. in an arrangement in which multiple denitrification and nitrification tanks are connected alternatingly one after another. It is also possible to implement alternating denitrification, i.e. a separation in time between nitrification and denitrification, by switching the delivery of oxygen or air off and on within the same tank. With the last two above-mentioned variants as well, it is optionally possible, by partial recycling of the wastewater or of the water/sludge mixture, to achieve largely complete nutrient breakdown, i.e. for example conversion of most of the nitrogen component of the nitrogen-containing components to dinitrogen (N₂), and thus improved wastewater purification.

A substantial disadvantage of the established methods for wastewater purification is their high energy consumption, which results chiefly from the high demand for delivery of air or oxygen for the oxidation reactions. In addition, the largely complete conversion of the nitrogen-containing compounds of nitrogen-containing substances, in the context of nitrification and denitrification, into largely inert dinitrogen that cannot be reutilized in terms of material or energy, represents a disadvantage of all established methods and processes for wastewater purification. The potential that exists for material- and energy-related utilization of the nitrogen components of nitrogen-containing substances, in particular of wastewaters such as those purified in sewage treatment plants by nitrification and denitrification, is thus not exploited. A further disadvantage is that in the context of the largely complete nitrification and largely complete denitrification in sewage treatment plants, very small proportions of gases hazardous to climate, such as dinitrogen monoxide, can occur as a byproduct because of poorly adjusted methods and undesired secondary reactions, and escape into the atmosphere. It is furthermore disadvantageous that at present, the wastewaters to be purified are not concentrated before entering the sewage treatment plant or, within the sewage treatment plant, before entering the entirely or partly anaerobic or aerobic stage. Large quantities of wastewater must therefore be treated, transported, and as applicable heated in sewage treatment plants. The result is a high level of energy consumption for wastewater transport, and in some cases for wastewater heating, within sewage treatment plants, as well as a large space requirement for sewage treatment plants.

Numerous studies and scientific efforts are focusing on the minimization of energy consumption. In order to improve the overall material balance and overall energy balance of sewage treatment plants, what is usually discussed is the material and energy utilization of the substances contained in the wastewaters. One example of this is, for example, the material- and energy-related utilization of the carbon-containing compounds of wastewaters resulting from the manufacture and energy-related utilization of sewage gas or biogas (CH₄). The application of more-recent methods for purifying nitrogen-containing wastewaters, such as the so-called ANAMMOX process, or the SHARON, BABE, or CANON processes, also offer opportunities to lower the energy consumption of sewage treatment plants. This is achieved by using alternative microorganisms, and by implementing a correspondingly adapted process control system and process sequence. In the context of discussions of the potential material-related utilization of the resources contained in wastewaters, there is also further discussion, for example, of utilizing phosphorus-containing wastewater constituents for fertilizer manufacture. With regard to the reduction of undesired climate-damaging emissions from sewage treatment plants, in particular the avoidance of dinitrogen monoxide emissions from sewage treatment plants based on incomplete nitrification or denitrification resulting from, for example, inaccurately adjusted processes as well as process errors and the occurrence of undesired secondary reactions, possibilities are now being discussed as to how these emissions can be avoided by way of appropriate microbiological and process-engineering and process control-engineering actions, and how to achieve nitrification and denitrification processes that proceed to completion. Utilization, in terms of material or energy, of the nitrogen components contained in the wastewaters is not, however, being discussed.

Nitrogen-containing compounds are therefore manufactured at present for the most part using technically and energetically demanding methods, in particular with the participation of dinitrogen as an inert educt, and/or successor products thereof. For example, dinitrogen monoxide, which is also referred to as nitrous oxide and is used e.g. as an oxidizing agent for combustion processes, for example in rocket motors, or as a narcosis agent, is generally manufactured at present by catalytic oxidation of ammonia, which is manufactured in particular from dinitrogen (N₂) and dihydrogen (H₂), or by thermal decomposition of ammonium nitrate. Manufacture is generally demanding in terms of energy and technology.

### BRIEF SUMMARY OF THE INVENTION

In the method according to the present invention for obtaining dinitrogen monoxide (N₂O or nitrous oxide) by microbiological or enzymatic processes from nitrogen-containing substances, the microorganisms, bacteria, archaea, eukaryotes, fungi, parasites, phages, cells, cell fractions or membrane fractions, and/or enzymes, and/or a combination thereof, that are to be used are selected, or manipulated or partly or entirely reversibly and/or irreversibly inhibited by suitable actions, or the corresponding microbiological or enzymatic processes are controlled, for example by way of suitable process conditions, in such a way that, in part or entirely, dinitrogen monoxide (N₂O) is formed from the nitrogen-containing compounds of the nitrogen-containing substances. Furthermore, the corresponding process conditions are selected so that the population of the correspondingly utilized microorganisms, bacteria, archaea, eukaryotes, fungi, parasites, phages, cells, cell fractions or membrane fractions, and/or enzymes, and/or a combination thereof that contribute to nitrous oxide production and/or to participating reaction sequences and/or to the treatment of nitrogen-containing substances is maintained to the greatest extent possible or elevated to the extent possible by propagation, and the reactions underlying nitrous oxide production and/or reaction sequences accompanying them and/or reactions or processes for the treatment of nitrogen-containing substances, proceed as completely and quickly as possible. Dinitrogen monoxide occurring in these reactions is separated out, captured, collected, if necessary purified, and/or conveyed to further processes, in particular combustion processes, e.g. methods for sewage gas and biogas combustion.

“Nitrogen-containing substances” for purposes of the present invention are, in particular, industrial and municipal wastewaters such as those purified in sewage treatment plants, or also liquids, solids, or sludges occurring in agriculture, for example liquid and solid manure, or also nitrogen-containing biomasses and wastes, in particular wastes or substances or wastewaters that occur, for example, in the context of biogas recovery.

An advantage of the method according to the present invention is that the manufacture of dinitrogen monoxide can be coupled, for example, with the treatment or purification of the nitrogen-containing substances, in particular in sewage treatment plants. For example, a favorable method for economically and energetically and technically undemanding recovery of dinitrogen monoxide can be implemented by way of suitable actions before, after, during, and/or instead of nitrification or denitrification in an aerobic and/or a partly or entirely anaerobic stage. Furthermore, by way of the utilization of this method and the utilization according to the present invention of the energy of dinitrogen monoxide, for example by combustion in facilities or processes for combusting sewage gas or biogas, the energy recovery of these methods or combustion processes can be increased, and the overall energy balance of sewage treatment plants and/or biogas plants can be improved. Furthermore, thanks to a suitable adaptation according to the present invention of the nitrification stage in sewage treatment plants or in processes for treating or purifying nitrogen-containing substances, the air or oxygen consumption, and thus also the energy consumption, of this stage, and thus the overall energy consumption of the process or sewage treatment plant, can be lowered. In addition, undesired dinitrogen monoxide emissions from sewage treatment plants, such as those that can occur at present because of process errors and undesired secondary reactions, can be very largely reduced or entirely avoided thanks to the encapsulation or gas-tight closure according to the present invention of the nitrification stage and/or denitrification stage, and optionally of further method stages. Because N₂O is a highly climate-damaging gas, these actions considerably reduce the climate impact of sewage treatment plants. A further advantage of the method according to the present invention is that as a result of the optional concentration according to the present invention of the nitrogen-containing substances, on the one hand a purified, water, as applicable potable water, can be obtained, and on the other hand the quantity of, for example, nitrogen-containing water or further nitrogen-containing substances to be treated can be reduced. The result is that, for example, the space and energy requirement of a sewage treatment plant, or of further processes for treating or purifying nitrogen-containing substances, can be reduced.

Within the methods used and established at present, as well as further ones discussed, for treating or purifying nitrogen-containing substances, in particular for purifying wastewaters, the nitrogen-containing substances, in particular the wastewaters to be treated, are as a rule not concentrated before entering the sewage treatment plant or before entering the aerobic or anaerobic stage of the sewage treatment plant. The nitrification or denitrification steps in sewage treatment plants are designed and controlled, microbiologically, enzymatically, and in terms of process engineering, so that the microbiological or enzymatic processes of nitrification and denitrification proceed, as described, as selectively and completely as possible. This means that in the context of nitrification, the intention is that maximally complete oxidation of the nitrogen-containing components occur, forming nitrate ions. In the context of denitrification, those nitrate ions are intended to be reduced as completely as possible to dinitrogen (N₂).

The method according to the present invention optionally contains, before and/or after and/or during the aerobic and/or the partly or entirely anaerobic process stage, an increase in the concentration of the nitrogen-containing substances, in particular of the industrial and municipal wastewaters. This can be implemented according to the present invention by a forward osmosis step. According to the present invention, the forward osmosis method can be implemented in particular so that the nitrogen-containing substance to be concentrated, e.g. the wastewater, is directed into or through one side of a tank and/or tube divided by a membrane, and/or through other flow geometries divided by a membrane. On the opposite side of the membrane, a so-called “draw solution” is directed into the tank and/or into the tube and/or into other flow geometries divided by a membrane, in or oppositely to the flow direction of the nitrogen-containing substance using the co-current or counter-current principle. Membranes based on polymeric materials, in particular based on polyesters and polyester fibers, or ceramic materials, can be used, for example, as suitable membrane materials. So-called “draw solutions” that can be used are, for example, aqueous solutions, suspensions, and/or mixtures of volatile or thermally unstable substances, in particular salts, in particular products of gaseous precursors such as, for example, CO₂ and/or NH₃ and/or water, such as e.g. an aqueous solution of NH₄HCO₃ and related as well as further substance systems. In addition, aqueous solutions, suspensions, and/or mixtures of magnetic substances, in particular aqueous suspensions of magnetic particles or aqueous solutions of water-soluble magnetic substances, for example solutions of iron-containing compounds, can be used. The composition and/or substance concentration of the corresponding draw solution is to be selected so that portions of the water of the nitrogen-containing substance, in particular of the wastewater, penetrate from the one side of the membrane through the membrane to the draw-solution side. As a result of the passage of water from the side of the nitrogen-containing substance, in particular the wastewater, to the draw-solution side, the nitrogen-containing substance becomes more concentrated, for example, in terms of its nitrogen content, and the draw solution becomes correspondingly diluted. The nitrogen-containing substance can then be conveyed to the further process steps according to the present invention, in particular to the process steps for dinitrogen monoxide production. The diluted draw solution is discharged and can be processed, for example by a suitable separation operation, in particular by magnetic separation of the magnetic substances and/or particles. What is produced thereby is largely pure water. The separated magnetic substances and/or particles can be used, by way of suitable method steps and with the use of water, to manufacture a draw solution suitable for forward osmosis. Depending on the composition of the draw solution, the diluted draw solution can also be processed by thermal decomposition of the thermolabile salts. According to the present invention it is possible to use for this, in particular, the combustion heat of sewage gas or biogas, or the combustion heat from combustion of the dinitrogen monoxide obtained according to the present invention along with sewage gas or biogas. The thermal energy required for this can be introduced into the draw solution by way of suitable technical implementations known to one skilled in the art. In this context, the corresponding gaseous precursors of the thermolabile salts, e.g. CO₂ and/or NH₃, escape, and largely pure water remains behind. The resulting gases can be introduced into portions of the diluted draw solution, or into water, in order to manufacture or regenerate the draw solution. The resulting draw solution, of sufficiently high concentration, can then be reused for the forward osmosis process. In the process steps that are part of forward osmosis in the broadest sense, the composition of the draw solution and/or of the nitrogen-containing substance can be analyzed using suitable measurement methods, in particular using conductivity measurements. The corresponding process steps can be correspondingly monitored and controlled by evaluating the correspondingly obtained measured values. The relative flow rates, in particular, can be adjusted in this context. Optional implementation of forward osmosis results not only in an increase in the concentration of the nitrogen-containing substances, with which, for example, a reduction in the space and energy requirements of the subsequent process steps can be achieved, but also in the recovery of fresh water, i.e. water that in this form can be reused and can, as applicable, be classified as potable water. The implementation of this optional process step appears useful chiefly in light of the present shortage of water as a resource.

### DETAILED DESCRIPTION OF THE INVENTION

According to the present invention, the recovery of dinitrogen monoxide (N₂O or nitrous oxide) via microbiological or enzymatic processes from nitrogen-containing substances, the microorganisms, bacteria, archaea, eukaryotes, fungi, parasites, phages, cells, cell fractions or membrane fractions, and/or enzymes, and/or a combination thereof are selected, or manipulated or inhibited partly or entirely reversibly and/or irreversibly by way of suitable actions, or the corresponding microbiological or enzymatic processes are controlled, for example by suitable process conditions, in such a way that, in part or entirely, dinitrogen monoxide (N₂O) is formed from the nitrogen-containing compounds of the nitrogen-containing substances. Moreover, the corresponding process conditions are selected so that the population of the correspondingly used microorganisms, bacteria, archaea, eukaryotes, fungi, parasites, phages, cells, cell fractions or membrane fractions, and/or enzymes, and/or a combination thereof that contribute to nitrous oxide production and/or to participating reaction sequences and/or to the treatment of nitrogen-containing substances is maintained to the extent possible or is increased if possible by propagation, and the reactions underlying nitrous oxide production and/or their accompanying reaction sequences and/or reactions or processes for treating nitrogen-containing substances proceed as completely and quickly as possible.

According to the present invention, the recovery of dinitrogen monoxide can be implemented as an accompaniment and/or a supplement to and/or instead of the previously implemented treatment or purification of nitrogen-containing substances, in particular the treatment or purification of wastewaters, in particular in sewage treatment plants, by means of nitrification and denitrification. In the context of an aerobic process step that can be viewed as a modification or variation of the nitrification presently applied in sewage treatment plants, the nitrogen-containing substances are brought into contact, by suitable actions, with oxygen or air and into contact with suitable microorganisms or heterotrophic as well as autotrophic bacteria, lithoautotrophic or chemolithoautotrophic microorganisms, and/or further microorganisms, bacteria archaea, eukaryotes, fungi, parasites, phages, cells, cell fractions or membrane fractions, and/or enzymes, and/or a combination thereof. Suitable according to the present invention in this context are, in particular, microorganisms, bacteria, archaea, eukaryotes, fungi, parasites, phages, cells, cell fractions or membrane fractions, and/or enzymes, and/or a combination thereof that, under aerobic conditions, convert nitrogen-containing substances partly or entirely into dinitrogen monoxide, or participate in the corresponding reaction sequences. These include, for example, nitrifiers such as Nitromonas europea and enzymes pertinent thereto. Also suitable, for example, are microorganisms, bacteria, archaea, eukaryotes, fungi, parasites, phages, cells, cell fractions or membrane fractions, and/or enzymes, and/or a combination thereof that, under aerobic conditions, convert nitrogen-containing substances partly or entirely into nitrite ions, or participate in corresponding reaction sequences. These likewise include, for example, nitrifiers such as Nitrosomonas europea. The formation of nitrate ions is possible according to the present invention in this process step, but not preferable.

The aerobic process step, in particular the delivery of oxygen or air, is controlled in this connection in such a way that oxidation of the nitrogen components results, at the highest possible proportion, in the formation of dinitrogen monoxide, and the gases formed are partly or entirely taken out of the liquid phase. With regard to the formation of nitrite and nitrate ions, the process step is controlled in such a way that the nitrite/nitrate ratio is maximized. The result is that the total oxygen consumption, and the energy consumption corresponding thereto, of this process step is minimized. Methods for blowing oxygen or air into wastewaters are known to one skilled in the art. Control of the process step, in particular of the delivery of oxygen or air and delivery of wastewater, as well as stipulation of the relevant flow rates and residence times, in particular of the liquid phase and/or sludge-containing phase, and stipulation of the pH and further process parameters, occur by way of the acquisition and evaluation of suitable measured data, in particular of the composition of the liquid phase, in particular with regard to the nitrate ion concentration and/or nitrite ion concentration and/or the concentration of further ions, in particular e.g. ammonium ions, and further substances, in particular e.g. the inhibitors to be used as applicable, the gas phase composition, in particular e.g. the dinitrogen monoxide concentration thereof, and the dissolved-oxygen concentration. When Nitrosomonas europea is used, for example, a dissolved-oxygen concentration of, for example O<5 mg/l, in particular O<2 mg/l, is useful for increased dinitrogen monoxide production. In addition, low pH values within this process step and/or within the wastewater leaving this process step are useful for the process described and the further process steps. pH values in the range from 3 to 10, in particular in the range from 5 to 9 and from 5 to 7, are useful for elevated dinitrogen monoxide production and a high nitrite/nitrate ion ratio.

The suitable microorganisms, bacteria, archaea, eukaryotes, fungi, parasites, phages, cells, cell fractions or membrane fractions, and/or enzymes, and/or a combination thereof can be present as a sludge, suspension, or the like, and can be transported with the aqueous medium, e.g. the wastewater to be purified. Optionally, they can also be immobilized on suitable supports using suitable methods. These supports are brought by suitable actions, in particular e.g. by flow-directing actions, into good contact with the nitrogen-containing substances. This has the advantage, for example, that the microorganisms, bacteria, archaea, eukaryotes, fungi, parasites, phages, cells, cell fractions or membrane fractions, and/or enzymes, and/or a combination thereof can be chosen and optimized selectively for this aerobic process step. It is furthermore thereby possible to purify and/or replace and/or remove the correspondingly occupied supports as necessary, and to regenerate them in an optional separate process step and/or, in a separate process step, select the reaction conditions in such a way that the selected microorganisms, bacteria, archaea, eukaryotes, fungi, parasites, and/or phages propagate as quickly as possible. It is further possible for the supports to be attached in such a way that they can be flushed alternatingly with various media, in particular for purification and/or regeneration and/or growth of the microorganisms, bacteria, archaea, eukaryotes, fungi, parasites, phages, cells, cell fractions or membrane fractions, and/or enzymes, and/or a combination thereof, and/or for further purposes. It is further possible, by skilful selection of the support and/or its geometry and/or the combination of the air or oxygen delivery system and/or inhibitor delivery system with the support, to improve contact, for example as a function of flow, among a nitrogen-containing substance, microorganisms, bacteria, archaea, eukaryotes, fungi, parasites, phages, cells, cell fractions or membrane fractions, and/or enzymes, and/or a combination thereof, and air or oxygen and/or optionally further usable substances, in particular suitable enzyme inhibitors.

In the context of a process step according to the present invention that is partly or entire anaerobic, which can be viewed as a modification or variation of the denitrification applied at present in sewage treatment plants, nitrogen-containing substances, in particular e.g. the nitrite ions and/or nitrate ions formed, as applicable, in part in the context of the aerobic process step and/or nitrogen monoxide present or formed in part, are brought by suitable actions into contact with suitable microorganisms or heterotrophic as well as autotrophic bacteria, lithoautotrophic or chemolithoautotrophic microorganisms, bacteria, archaea, eukaryotes, fungi, parasites, phages, cells, cell fractions or membrane fractions, and/or enzymes, and/or a combination thereof. Suitable according to the present invention in this context are, in particular, microorganisms, bacteria, archaea, eukaryotes, fungi, parasites, phages, cells, cell fractions or membrane fractions, and/or enzymes, and/or a combination thereof that, under the selected conditions, partly or entirely convert nitrogen-containing substances, in particular nitrite ions and nitrate ions, or nitrogen monoxide formed in part, into dinitrogen monoxide, or that participate in corresponding reaction sequences. These include, for example, denitrifiers that do not have the capability for N₂O reductase or whose capability for N₂O reductase can be partly or entirely inhibited. These include, for example, Agrobacterium tumefaciens, Alcaligenes faecalis, Alcaligenes spp., Agrobacterium tumefaciens, Pseudomonas chlororaphis, Pseudomonas perfectomarinus, Pseudomonas fluorescens, Pseudomonas caryophylli, Pseudomonas aureofaciens, Pseudomonas aerogenes, Pseudomonas spp., Propionibacterium acidipropionici, Neisseria spp., Neisseria sicca, Neisseria flavescens, Neisseria sasflava, Neisseria mucosa, Bacillus licheniformis, Chromobacterium violaceum, Chromobacterium lividum, Corynebacterium nephridii, Thiosphaera pantotropha, Thiobacillus denitrificans, Paracoccus halodenitrificans, Achromobacter cycloclastes, and some species of Rhizobia, as well as species of nitrifying or denitrifying fungi such as, for example the fungus Fusarium oxysporum. The microorganisms and bacteria also used hitherto in denitrification can also be correspondingly utilized. The partial formation of dinitrogen (N₂) in this process step is possible according to the present invention but not preferable.

The partly or entirely anaerobic process step is controlled, in this connection, so that conversion of the nitrogen-containing components, in particular reduction of the nitrite ions and nitrate ions and of nitrogen monoxide present or formed in part, results at the highest possible proportion in the formation of dinitrogen monoxide, and the gas formed is taken partly or entirely out of the liquid phase. Control of the process step, in particular the delivery of wastewater and/or sludge, as well as stipulation of the relevant flow rate and residence time in the current and/or preceding and/or downstream process step, occur by way of the acquisition and evaluation of suitable measured data, in particular of the composition of the liquid phase, in particular with regard to the dissolved-oxygen concentration and/or nitrate ion concentration and/or nitrite ion concentration and/or the concentration of further ions and further substances, in particular e.g. the inhibitors to be used as applicable, and/or the C/N ratio, the pH, the gas phase composition, in particular e.g. its dinitrogen monoxide concentration and/or its dinitrogen monoxide concentration and/or its concentration of inhibitors to be used as applicable, and/or temperature. If the microorganisms, bacteria, archaea, eukaryotes, fungi, parasites, phages, cells, cell fractions or membrane fractions, and/or enzymes, and/or a combination thereof that are used have the capability for N₂O reductase, this can thus likewise be partly or entirely inhibited according to the present invention by controlling suitable actions. This occurs in particular when using, for example, of Thiosphaera pantotropha, Thiobacillus denitrificans, Paracoccus halodenitrificans, and Achromobacter cyclolastes, in particular by targeted adjustment of the oxygen content of the partly or entirely anaerobic process step by the delivery of oxygen or air. Methods for blowing oxygen or air into wastewaters are known to one skilled in the art. When Thiosphaera pantotropha, Thiobacillus denitrificans, Paracoccus halodenitrificans, and Achromobacter cyclolastes are used, for example, a dissolved-oxygen concentration in the range of a 0- to 90-percent oxygen saturation of the liquid phase, in particular a 0- to 25-percent oxygen saturation of the liquid phase, is useful for elevated dinitrogen monoxide production. Selection of the optimum oxygen saturation for dinitrogen monoxide production depends very strongly on the microorganisms, bacteria, archaea, eukaryotes, fungi, parasites, phages, cells, cell fractions or membrane fractions, and/or enzymes, and/or a combination thereof that are involved, as well as all the surrounding process conditions. Analogously to the oxygen concentration, according to the present invention the carbon content of the wastewater or the C/N ratio of the nitrogen-containing substance can be controlled so that elevated dinitrogen monoxide production is achieved. C/N ratios and/or COD/NO₃—N ratios and/or COD—NO₂—N ratios less than 10, in particular less than 5 or less than 3, are useful for this. These can be established, for example, by adding carbon-rich wastewater or by regulated adjustment of the relative residence times of the nitrogen-containing substances in the respective process steps. Low pH values within this process step and/or within the wastewater leaving this process step are furthermore useful for the process described and the further process steps. pH values in the range from 3 to 10, in particular in the range from 5 to 9 and from 5 to 7 are useful for elevated dinitrogen monoxide production.

In addition, before and/or during the aerobic or the partly or entirely anaerobic process stage, according to the present invention a complete or partial inhibition of dinitrogen monoxide reductase can occur using suitable irreversible and/or reversible, or non-competitive and/or competitive inhibitors, and/or by substrate inhibition or product inhibition and/or by the addition of precursors of corresponding inhibitors. Irreversible and/or reversible, or non-competitive and/or competitive inhibitors suitable for the method for obtaining dinitrogen monoxide are, for example, substances that deactivate the active center of dinitrogen monoxide reductase or bond to that center instead of dinitrogen monoxide. Substances suitable in this connection are substances that, for example, have a structural similarity to dinitrogen monoxide or acetylene, for example N₂O-containing metal complexes. Solid, gaseous, or liquid substances can act as inhibitors of N₂O reductase capability. These include, in particular, acetylene, ethene, azides, carbides, cyanides, 2,4-dinitrophenol, monoiodoacetate, CuSO₄, and CO, as well as compounds that exhibit these functionalities in their molecular structure and can thus function comparably as inhibitors. Alternatively, an acoustically based modification of the cells of the corresponding microorganisms, bacteria, archaea, eukaryotes, fungi, parasites, phages, cells, cell fractions or membrane fractions, and/or a combination thereof, in particular of Ps. denitrificans, can contribute to increased dinitrogen monoxide production.

The suitable microorganisms, bacteria, archaea, eukaryotes, fungi, parasites, phages, cells, cell fractions or membrane fractions, and/or enzymes, and/or a combination thereof can in turn be present as a sludge, suspension, or the like, and can be transported with the aqueous medium, e.g. the wastewater to be purified. Optionally, they can be immobilized on suitable supports using the methods described. According to the present invention, for example, the cytoplasmic membrane fraction of the marine denitrifier Pseudomonas perfectomarinus, or resting cells of Corynebacterium nephridii, can be immobilized and can contribute to dinitrogen monoxide production under partly or entirely anaerobic conditions. It is furthermore thereby possible to selectively immobilize N₂O reductase-inhibited microorganisms, bacteria, archaea, eukaryotes, fungi, parasites, phages and/or phages and/or alternatively the combination of the enzymes nitrate reductase, nitrite reductase, and NO reductase, on a support, bring it into contact with nitrogen-containing substances, in particular nitrite ion-containing and nitrate ion-containing wastewaters, and thereby implement a favorable method for obtaining N₂O. It is further possible, by skilful selection of the support and its geometry, to improve contact, for example as a function of flow, among the nitrogen-containing substance, microorganisms, bacteria, archaea, eukaryotes, fungi, parasites, phages, and/or enzymes, and air or oxygen, or optionally further usable substances, in particular suitable enzyme inhibitors and/or a combination of the support systems with the addition of air or oxygen and/or inhibitors or precursors thereof.

In order to prevent or limit the reduction reaction of dinitrogen monoxide to nitrogen, according to the present invention copper ions of the metalloenzyme used for reduction of the dinitrogen monoxide can alternatively be reduced, removed, or complexed before and/or during the aerobic or the partly or entirely anaerobic process stage. Alternatively, a copper separation by way of selective ion exchangers can also occur for purposes of the invention before and/or during the aerobic or the partly or entirely anaerobic process stage. Removal and/or complexing of the copper ions can occur, before and/or during the aerobic or the partly or entirely anaerobic process stage, for example by the use of complexing agents, by reduction using suitable metals or metal ions, and by way of all redox systems that can entirely or partly reduce copper ions at the existing concentration, using selective ion exchangers or by electrochemical reduction, for example by electrolysis.

Suitable complexing agents for copper ions are, for example, chelate-forming substances, for example tetraacetylethylenediamine (TAED). Also suitable, however, as complexing agents for removing copper ions are, for example, sulfonamide-substituted thiono ligands, ligands analogous to 1-(chloro-3-indolylazo)-2-hydroxynaphthalene-3,6-disulfonic acid, or chlorophyll-based ligands.

Suitable metals that can be used to reduce copper ions, for example by sedimentation, are, for example, iron, tin, and zinc. Suitable metal ions are, for example Sn²⁺ ions. Suitable further redox systems for reducing copper ions are, for example, nitrate and/or nitrite ions at suitable concentration ratios. Iron is particularly preferred.

In order to remove copper ions by the use of selective ion exchangers, it is known to one skilled in the art to use ion exchangers selective for copper ions. Suitable ion exchangers are, for example, those that contain metal ions, for example calcium, magnesium, or sodium ions, as exchange ions, as well as furthermore chelate-forming and adsorptive ion exchangers. Suitable ion exchangers are, for example, modified sulfonated polystyrene ion exchangers, variously substituted iminodiacetic acid ion exchangers, and further polymer- or silicate-based ion exchangers.

Ion exchangers can also be used, for purposes of the present invention, as immobilizers of the microorganisms, bacteria, archaea, eukaryotes, fungi, parasites, phages, or cells, cell fractions or membrane fractions, and/or enzymes, and/or a combination thereof. The microorganisms, bacteria, archaea, eukaryotes, fungi, parasites, phages, or cells, cell fractions or membrane fractions, and/or enzymes, and/or a combination thereof are, in that context, immobilized e.g. by complexing of the copper ions. Copper complexing allows an inhibiting effect on dinitrogen monoxide reductase, and thus elevated N₂O recovery, to be achieved.

The complexing agents or the metals or metal ions used for sedimentation, further redox systems, and ions or ion exchangers, as well as suitable irreversible and/or reversible, or non-competitive and/or competitive inhibitors, can be added to the liquid phase, for example, before and/or during the aerobic or the partly or entirely anaerobic process stage, in liquid, solid, or gaseous form, granulate form, and/or sheet form.

It is alternatively possible, however, for example, in particular when an ion exchanger is used to remove copper ions and/or to immobilize the bacteria, archaea, eukaryotes, fungi, parasites, phages, or cells, cell fractions or membrane fractions, and/or enzymes, and/or a combination thereof, to direct the nitrogen-containing substance, in particular the wastewater, through a suitable column that contains the ion exchanger. The ion exchanger or the ion exchange material can be present in this case as a structured or disordered packing. It is possible, for example, for the ion exchanger to be contained in the column in the form of a woven or knitted fabric, or also as packing elements. It is also possible to dispense a granulated ion exchanger into the column. An advantage of using a column is that regeneration of the ion exchanger is possible in simple fashion, for example by replacement or by switching over to a second column that likewise contains an ion exchanger. The ion exchanger and/or the immobilized bacteria, archaea, eukaryotes, fungi, parasites, phages, or cells, cell fractions or membrane fractions, and/or enzymes, and/or a combination thereof in the unused column can then be regenerated, and/or the growth thereof can be stimulated.

The execution sequence of the aerobic and partly or entirely anaerobic stage is not obligatorily defined. Similarly to the case with the established methods, all variants described in the existing art, i.e. first a partly or entirely anaerobic stage and then an aerobic stage, or first an aerobic stage and then a partly or entirely anaerobic stage, as well as a stage alternating over time between aerobic and partly or entirely anaerobic, as well as implementation of a tank cascade made up of correspondingly designed aerobic and partly or entirely anaerobic tanks, are possible. It is therefore possible to modify existing methods for wastewater purification in accordance with the present invention, and thereby to implement simple and favorable N₂O recovery from wastewaters. This also applies in particular to the combination, optional according to the present invention, of the recovery according to the present invention of N₂O with an anaerobic wastewater treatment in the context of which carbon-containing components of the wastewater are converted partly or entirely to methane.

Maximally complete conversion of the nitrogen-containing substances into dinitrogen monoxide can be achieved in all process step sequence variants and/or all process step combinations with further methods for treating nitrogen-containing substances, in particular aerobic and anaerobic wastewater treatment, by optionally recycling the wastewater or the water/sludge mixture.

The component of the substance mixture that cannot be recycled within the process step variants can, by analogy with the established methods, be conveyed to a sedimentation process or to further methods for sludge separation, e.g. membrane-based processes. It is moreover alternatively possible according to the present invention for the process steps according to the present invention for obtaining N₂O and/or the process steps for separating the dinitrogen monoxide from the aqueous phase and/or the methods for sludge separation to be preceded or followed by another, i.e. unmodified, method for nutrient breakdown, e.g. unmodified complete aerobic and anaerobic process steps for nitrification or denitrification. It is thereby possible to ensure that the requirements stipulated for the finally purified water, for example in terms of maximum contaminant concentrations and maximum nitrogen contents and dinitrogen monoxide contents, are complied with. In addition, a forward osmosis step can optionally according to the present invention, as described above, precede and/or be interposed between and/or follow all the above-described process sequence variants. Further quantities of largely pure water are thereby obtained.

According to the present invention, the process steps selected for obtaining dinitrogen monoxide can be monitored using suitable sensors and/or suitable methods, and controlled by way of suitable actions. Methods for monitoring the process steps are, for example, gas-sensor methods. For example, the composition of the gas phase can be analyzed by gas-spectroscopy measurements, in particular based on FTIR or laser spectroscopy. The composition of the aqueous phase can be analyzed, for example, using spectroscopic as well as potentiometric or coulombometric methods, and the use of suitable electrodes, for example in order to determine pH, as well as further methods. Further gas-analysis processes and methods for analyzing the composition of the aqueous phase are known to one skilled in the art. By tracking, evaluating, and utilizing the measured values thereby obtainable, it is possible to control the process described according to the present invention, as well as established methods for the treatment or purification of nitrogen-containing substances using suitable actions, for example by regulating the temperature, the pH (which can also be regulated, in particular, by regulating the delivery of oxygen or air), the addition of carbon-rich wastewaters or additional carbon components, in particular e.g. organic solvent wastes, as well as the relative residence times of the nitrogen-containing substances and of the microorganisms, bacteria, archaea, eukaryotes, fungi, parasites, phages, cells, cell fractions or membrane fractions, and/or enzymes, and/or a combination thereof, and of the sludge, and/or the flow rates and inhibitor addition before/after or during the individual process steps, so as to achieve a maximization of the recovery of dinitrogen monoxide, a reduction in the energy consumption of the process steps, and a high-quality purified wastewater. More reliable operation of the underlying reactions and process steps, in particular avoidance of emissions of climate-damaging N₂O, is furthermore ensured thereby.

For the recovery of dinitrogen monoxide, it is preferred to separate it from the liquid phase. A portion of the dinitrogen monoxide formed is discharged from the aqueous phase in the context of the blowing in of air or oxygen in the aerobic or the partly or entirely anaerobic stage. To ensure that the dinitrogen monoxide discharged in this fashion can be utilized, and cannot enter the atmosphere and act in climate-damaging fashion therein, it is necessary according to the present invention to encapsulate in gas-tight fashion the corresponding process stages in which dinitrogen monoxide escapes from the liquid phase. Possibilities for gas-tight encapsulation of the process steps are known to one skilled in the art, for example, from biogas applications and further industrial gas-based processes.

In addition to the partial or complete discharge, in the context of the blowing in of air or oxygen in the aerobic or the partly or entirely anaerobic stage, of the dinitrogen monoxide that is formed, it is permissible according to the present invention, for example before, during, and/or after the aerobic or the partly or entirely anaerobic stage, to utilize active gas extraction in order to separate the dinitrogen monoxide from the liquid phase. For this it is possible, for example, to apply a gas-tight cover and carry out aspiration via negative pressure. Further portions of the dinitrogen monoxide dissolved in the liquid phase can thereby be transferred into the gas phase. In addition to the application of negative pressure, dinitrogen monoxide dissolved in the liquid phase can also be separated out, for example, by pressure variation.

It is also possible to separate dinitrogen monoxide dissolved in the liquid phase from the liquid phase, for example, by salting out, stripping, or expulsion with a gas, for example with air, oxygen, or steam, or also with media different therefrom that are known to one skilled in the art, before, during, and/or after the aerobic or the partly or entirely anaerobic stage.

It is alternatively possible, during and/or after the aerobic or the partly or entirely anaerobic stage, also to transfer the dinitrogen monoxide into the gas phase, for example, by the introduction of thermal energy. The introduction of thermal energy lowers the solubility of dinitrogen monoxide in the liquid. In addition, a portion of the liquid evaporates. The introduction of thermal energy can be carried out by way of any method known to one skilled in the art. The thermal energy is usually implemented by heating with a suitable heat exchanger or an electrical heater. If a heat exchanger is used, it is then possible on the one hand, for example, to use a container having a double jacket, the double jacket being heated. Alternatively, however, any kind of heat exchange element can also be provided in the container in which the dinitrogen monoxide-containing liquid is contained. Such heat exchange elements are, for example, heat exchange plates or tubes through which a heat transfer medium flows. Heat transfer media that are usually used are, for example, heat transfer oils, water, or steam.

In addition, further methods for separating the dinitrogen monoxide from the liquid phase, for example the application of thin-film evaporators or thin-layer reactors, in which gaseous constituents preferentially leave the liquid phase thanks to implementation of a thin film of liquid, can be applied before, during, and/or after the aerobic or the partly or entirely anaerobic stage. According to the present invention, microorganisms, bacteria, archaea, eukaryotes, fungi, parasites, phages, cells, cell fractions or membrane fractions, and/or enzymes, and/or a combination thereof that are suitable according to the present invention can optionally be immobilized on these thin-film evaporators and/or thin-layer reactors.

In order to achieve an improved yield of dinitrogen monoxide, it is furthermore possible, for example before separation of the dinitrogen monoxide from the liquid phase, to carry out a concentration of the dinitrogen monoxide, for example by extraction or further, for example, membrane-based methods.

As a result of the separation of dinitrogen monoxide from the liquid phase it is possible, depending on the method utilized, to obtain pure dinitrogen monoxide or a gas mixture enriched in dinitrogen monoxide. The purity of the dinitrogen monoxide is dependent in this context on the type of treatment and separation. “Gaseous phase” or “waste gas” refers in this connection to all gaseous products that occur in the context of the recovery according to the present invention of dinitrogen monoxide. If the dinitrogen monoxide is produced in the context of wastewater purification in sewage treatment plants, then depending on process conditions the gaseous phase can also contain, alongside dinitrogen monoxide, for example gaseous hydrocarbons, carbon monoxide, carbon dioxide, and ambient air constituents as applicable. Further gaseous breakdown products of wastewater purification, as well as any inhibitors that may have been used, precursors thereof, and/or reaction products, can also be contained in the waste gas.

In an embodiment, the dinitrogen monoxide is, for example, separated out from the waste gas by way of a gas membrane selective for dinitrogen monoxide. Alternatively, it is also possible to use a gas membrane that is impermeable to dinitrogen monoxide and that allows other constituents of the dinitrogen monoxide-containing gas to pass through, and in that way to increase the concentration of dinitrogen monoxide in the retentate stream. In addition, depending on the membrane used, gaseous enzyme inhibitors or reaction products thereof or precursors thereof can be separated out from the gas stream and reutilized according to the present invention. Such gas membranes selective for dinitrogen monoxide or for further gases are known to one skilled in the art. Membranes based on sulfonate-containing aromatic polyamides and poly-N-vinylamides can be used in particular, and also Lestosil membranes, cellulose-based, in particular cellulose acetate membranes, as well as silicone-based, polydimethylsiloxane-based, and poly[bis(trifluoroethoxy)phosphazenene] as well as further and related membranes and membrane systems.

It is further also possible, however, for example, for the dinitrogen monoxide of the waste gas to be liquefied, for example by an increase in pressure or a decrease in temperature. The liquefied dinitrogen monoxide condenses out and can be collected.

Other gas purification methods known to one skilled in the art can also be used to separate the dinitrogen monoxide from the waste gas. Such methods are, for example, stripping, membrane, condensation, adsorption, distillation, or rectification processes and/or further known methods for separating and purifying gases. Separation of the dinitrogen monoxide using suitable molecular sieves, for example, by introducing and dissolving the dinitrogen monoxide-containing gas into liquid or solid media for concentration, or selective adsorption processes, are suitable. Suitable liquid or solid media through which the dinitrogen monoxide-containing gas is directed are, for example, an iron sulfate solution and iron sulfate emulsified in sulfuric acid, as well as P₂O₅. A rectification, distillation, or extraction process can then follow for further purification.

It is also possible according to the present invention, however, for the dinitrogen monoxide to be used in unpurified form, depending on its further use.

In addition to the recovery of dinitrogen monoxide from the purification of wastewaters, it is also possible according to the present invention to obtain dinitrogen monoxide using microbiological or enzymatic processes from nitrogen-containing substances, in the context of any desired further processes. It is thus also possible, for example, to obtain dinitrogen monoxide from nitrogen-containing substances or liquids that occur, for example, in the context of biogas recovery. Alongside household wastewaters, it is moreover possible also to use household wastes, wastewaters, wastes, and waste and other substances that occur in industry and agriculture, in particular cereals and/or grass clippings, for dinitrogen monoxide recovery. Dinitrogen monoxide can thus also be recovered, for example, from liquid manure, fermentation residues of biogas facilities, compost, manure, and industrial wastewaters from, for example, dairy operations and slaughterhouses.

The dinitrogen monoxide recovered by way of the method according to the present invention can be conveyed to an oxidation reaction or to combustion processes as an oxygen carrier. The dinitrogen monoxide can be used, for example, for the combustion of coal, natural gas, biogas and sewage gas, as well as fuels, in internal combustion engines, cogeneration power plants, or in fuel cells. The conveyance of dinitrogen monoxide into combustion processes improves the energy content and the efficiency, and thus the maximally usable energy, of combustion processes as compared with the use of air as an oxygen carrier. The result is that the energy efficiency of internal combustion engines, cogeneration power plants, or fuel cells is considerably improved, and energy-specific carbon dioxide emissions are reduced. According to the present invention, the corresponding processes for N₂O utilization are monitored using gas sensors so as thereby to avoid undesired emission of N₂O and of further pollutants such as, for example, NO and NO₂ that can occur, for example, in combustion processes. Gas sensors suitable for this are known to one skilled in the art. If applicable, further actions for waste gas purification are taken according to the present invention, for example the implementation of corresponding waste gas catalysts such as those known from industrial and automotive applications.

A particularly suitable use of the dinitrogen monoxide obtained according to the present invention is conveyance of the dinitrogen monoxide obtained in sewage treatment plants to processes in which sewage gas obtained by sludge digestion or in further anaerobic methods is exploited for energy or combusted. In addition to the increase in power generation thereby obtained, the resulting combustion heat can be used in various ways. As described, the heat can be used in the context of the optionally usable concentration process by forward osmosis, for example for thermal decomposition of the thermolabile salts and thus for treatment of the draw solution and for water recovery. The heat can further be fed into a district heating network or used to heat the wastewater, in particular the optionally concentrated wastewater. Methods for media heating using thermal energy are sufficiently known to one skilled in the art. The resulting increase in the temperatures of the wastewater undergoing purification causes an acceleration of microbiological or enzymatic processes. The maximum capacity of a sewage treatment plant is thereby increased, or the specific area or volume requirement of the respective process steps per population equivalent is decreased. Alternatively, the thermal energy can be used for dinitrogen monoxide separation from the liquid phase, or for methods for purifying the gas. The heat can furthermore be utilized to accelerate the growth or regeneration of the microorganisms, bacteria, archaea, eukaryotes, fungi, parasites, phages, cells, cell fractions or membrane fractions, and/or enzymes, and/or a combination thereof that are used. Alternatively, the anaerobic process steps used for sewage gas manufacture can be heated and thus accelerated. It is moreover possible to convert the thermal energy into cooling energy. Methods used for this, such as e.g. the utilization of absorption cooling systems, in particular in conjunction with heat and cold reservoirs, are known to one skilled in the art. The resulting heat or cold can thus be conveyed, for example, to accompanying industrial processes such as, for example, the pasteurization of milk, the cooling of cooling areas and storage areas in the dairy and meat-packing industry, and further processes, and to municipalities and industries in order to heat and cool buildings. Partial or complete implementation of the aforementioned actions thus, in sum, considerably improves the overall energy balance of sewage treatment plants and accompanying processes, for example industrial process steps, as well as their climate relevance.

Further suitable applications of dinitrogen monoxide are also utilization as an educt of a conversion reaction or further chemical syntheses.

The recovery according to the present invention of dinitrogen monoxide allows the chemical energy of nitrogen-containing substances, in particular nitrogen-containing wastewaters, to be utilized in favorable fashion. The utilization of wastewaters in terms of energy technology has hitherto been limited to the recovery of biogas or hydrogen on the basis of the organic carbon compounds contained in the wastewater. The method according to the present invention for recovering dinitrogen monoxide opens up a new approach to the utilization of wastewater in terms of energy technology, based on nitrogen-containing components contained in the wastewater, and implementation thereof furthermore results in a considerable improvement in the energy and climate balance of sewage treatment plants.

1-24. (canceled)

25. A method for obtaining dinitrogen monoxide, comprising:
implementing one of a microbiological or an enzymatic process using a nitrogen-containing substance including at least one of microorganisms, bacteria, archaea, eukaryotes, fungi, parasites, phages, cells, cell fractions, membrane fractions, and enzymes to produce dinitrogen monoxide.

26. The method as recited in claim 25, wherein the nitrogen-containing substance includes at least one of: wastewaters purified in sewage treatment plants; compounds containing at least one of ammonium ions, ammonium compounds, nitrite ions, nitrate ions, ammonium groups, nitrite groups, and nitrate groups; liquid manure; solid manure; nitrogen-containing biomasses; and nitrogen-containing wastes.

27. The method as recited in claim 25, wherein the at least one of the microorganisms, bacteria, archaea, eukaryotes, fungi, parasites, phages, cells, cell fractions, membrane fractions, and enzymes is selected so that at least one of (i) dinitrogen monoxide and (ii) a corresponding precursor or an intermediate product is formed from a nitrogen-containing compound of the nitrogen-containing substance.

28. The method as recited in claim 25, wherein the at least one of the microorganisms, bacteria, archaea, eukaryotes, fungi, parasites, phages, cells, cell fractions, membrane fractions, and enzymes is at least one of manipulated and influenced in such a way that at least one of (i) dinitrogen monoxide and (ii) a corresponding precursor or an intermediate product is formed from a nitrogen-containing compound of the nitrogen-containing substance.

29. The method as recited in claim 28, wherein:
the implementing of one of the microbiological or the enzymatic process includes at least partly anaerobic process stages; and
recycling of the nitrogen-containing substance takes place between the at least partly anaerobic process stages.

30. The method as recited in claim 28, wherein conditions of the one of the microbiological or enzymatic process are selected so that: (i) the population of the at least one of the microorganisms, bacteria, archaea, eukaryotes, fungi, parasites, phages, cells, cell fractions, membrane fractions, and enzymes contributing to production of the at least one of dinitrogen monoxide and the corresponding precursor or the intermediate product is one of maintained or increased by propagation; and (ii) the speed and completeness of the one of the microbiological or enzymatic process for the production of the at least one of dinitrogen monoxide and the corresponding precursor or the intermediate product are maximized.

31. The method as recited in claim 29, wherein copper ions are at least one of reduced, complexed, separated out, and exchanged at least one of before, after, and during at least partly anaerobic process.

32. The method as recited in claim 31, wherein the copper ions are at least one of complexed, reduced, and removed by reduction using at least one of selected metals, redox systems, and ions, by using at least one of selective ion exchangers and an electrochemical reaction.

33. The method as recited in claim 29, wherein activity of dinitrogen monoxide reductase is one of limited or suspended at least one of before, after, and during the at least partly anaerobic process stages, by at least one of a selected inhibitor, a substrate inhibition, product inhibition, regulation of the pH, regulation of oxygen content, regulation of temperature, and regulation of C/N ratio of the nitrogen-containing substance.

34. The method as recited in claim 33, wherein at least one of the pH, the oxygen content, the temperature, the C/N ratio of the nitrogen-containing substance, and a flow-through rate of the at least one of the microorganisms, bacteria, archaea, eukaryotes, fungi, parasites, phages, cells, cell fractions, membrane fractions, and enzymes is used as at least one of a regulated variable and a regulating variable for the implementing of the one of the microbiological or the enzymatic process.

35. The method as recited in claim 29, wherein the at least one of the microorganisms, bacteria, archaea, eukaryotes, fungi, parasites, phages, cells, cell fractions, membrane fractions, and enzymes is coordinated with respective process stages.

36. The method as recited in claim 29, wherein the at least one of the microorganisms, bacteria, archaea, eukaryotes, fungi, parasites, phages, cells, cell fractions, membrane fractions, and enzymes is immobilized on at least one selected support configured as at least one of a porous material, an ion exchanger, a thin-film evaporator, and a thin-layer reactor.

37. The method as recited in claim 36, wherein at least one of:
(i) wherein the at least one selected support is configured to be at least one of replaced, cleaned, and regenerated;
(ii) wherein the at least one selected support is configured to be mounted in such a way that the at least one selected support is flushed alternatingly with different media; and
(iii) wherein the at least one selected support is configured so that, upon introduction of at least one of oxygen, inhibitor, and inhibitor precursor, good contact is enabled between (a) the at least one of the introduced oxygen, inhibitor, and inhibitor precursor, and (b) the at least one of the microorganisms, bacteria, archaea, eukaryotes, fungi, parasites, phages, cells, cell fractions, membrane fractions, and enzymes.

38. The method as recited in claim 29, wherein the at least one of the microorganisms, bacteria, archaea, eukaryotes, fungi, parasites, phages, cells, cell fractions, membrane fractions, and enzymes is contained in at least one of liquid and gaseous phase, and wherein the dinitrogen monoxide produced in the one of the microbiological or the enzymatic process is separated from the at least one of the microorganisms, bacteria, archaea, eukaryotes, fungi, parasites, phages, cells, cell fractions, membrane fractions, and enzymes at least one of before and after the at least one of the microorganisms, bacteria, archaea, eukaryotes, fungi, parasites, phages, cells, cell fractions, membrane fractions, and enzymes is separated from the at least one of the liquid and gaseous phase.

39. The method as recited in claim 38, wherein the dinitrogen monoxide produced in the one of the microbiological or the enzymatic process is separated from the at least one of the microorganisms, bacteria, archaea, eukaryotes, fungi, parasites, phages, cells, cell fractions, membrane fractions, and enzymes by a selective membrane process.

40. The method as recited in claim 38, wherein process tanks for the one of the microbiological or the enzymatic process are encapsulated in gas-tight enclosure, and wherein the dinitrogen monoxide is separated by the gas-tight enclosure and conveyed for a further process including at least one of combustion process and a catalytic gas purification process.

41. The method as recited in claim 40, wherein the dinitrogen monoxide obtained is conveyed to a further reaction as at least one of an oxygen carrier and a nitrogen carrier.

42. The method as recited in claim 41, wherein the further reaction is at least one of (i) a combustion reaction of at least one of coal, natural gas, sewage gas, biogas, and fuel, and (ii) a reaction in a fuel cell.

43. The method as recited in claim 42, wherein at least one of electricity and thermal energy obtained from the further reaction is conveyed to at least one of a power grid and a thermal heating network.

44. The method as recited in claim 40, wherein the dinitrogen monoxide obtained is conveyed as an educt at least one of (i) to a conversion reaction, and (ii) for further synthesis.

45. The method as recited in claim 29, wherein the nitrogen-containing substance is concentrated in a concentrating process at least one of before, during, and after the at least partly anaerobic process stages.

46. The method as recited in claim 45, wherein the concentrating process for the nitrogen-containing substances is at least one of:
a forward osmosis process using (i) at least one of a flow geometry divided by a membrane, a tank divided by a membrane, and a tube divided by a membrane, and (ii) at least one of a draw solution using a thermally unstable substance formed from at least one gaseous precursor including CO2 and NH3, a draw solution utilizing a magnetic substance;
monitored by a conductivity measurement to analyze the composition of at least one of the draw solution and the nitrogen-containing substance; and
regulated, based on the conductivity measurement, by controlling a flow rate of the at least one of the draw solution and the nitrogen-containing substance.

47. The method as recited in claim 38, wherein the method for obtaining dinitrogen monoxide is implemented as one of a supplement to, or a substitution of, a process for purification of the nitrogen-containing substance.

48. The method as recited in claim 38, wherein the portion of media separated from the dinitrogen monoxide is conveyed to a further process including at least one of unmodified aerobic process steps and anaerobic process steps for one of nitrification or denitrification for nutrient breakdown.
